(19) United States
(12) Patent Application Publication
(10) Pub. No.: US 2015/0141807 A1
(43) Pub. Date: May 21, 2015

(54) REAL-TIME IN VIVO MEASUREMENT OF THE 3D ANGULAR ORIENTATION OF CARDIOVASCULAR STRUCTURES

(71) Applicant: Mayo Foundation for Medical Education and Research, Rochester, MN (US)

(72) Inventor: Kenneth A. Fetterly, Spring Valley, MN (US)

(21) Appl. No.: 14/400,391

(22) PCT Filed: Mar. 8, 2013
(86) PCT No.: PCT/US2013/029996

§ 371 Date: Nov. 11, 2014

Related U.S. Application Data
(60) Provisional application No. 61/645,822, filed on May 11, 2012.

(51) Int. Cl. A61M 25/01; A61M 25/10; A61B 6/00; A61B 6/12

(52) U.S. Cl. CPC A61M 25/0108; A61B 6/12; A61M 25/10; A61B 6/52; A61B 6/481; A61B 6/4028
USPC 600/424

(57) ABSTRACT

This document provides materials and methods for determining three-dimensional spatial orientations of blood vessels, cardiac valves, and other anatomical structures within a mammal during a clinical procedure. For example, materials and methods for determining the three-dimensional spatial location, orientation, and size of a cardiac valve within a mammal during a trans-catheter cardiac valve implantation or replacement procedure are provided.

### CROSS-REFERENCE TO RELATED APPLICATIONS

This application claims the benefit of U.S. Provisional Application Ser. No. 61/645,822, filed May 11, 2012. The disclosure of the prior application is considered part of (and is incorporated by reference in) the disclosure of this application.

### BACKGROUND

1. Technical Field

This document relates to materials and methods for determining three-dimensional spatial orientations of blood vessels, cardiac valves, and other anatomical structures within a mammal during a clinical procedure. For example, this document provides materials and methods for determining the three-dimensional spatial location, orientation, and size of a cardiac valve within a mammal during a trans-catheter cardiac valve implantation or replacement procedure.

2. Background Information

X-ray angiography imaging, used to guide clinical procedures performed in invasive cardiac laboratories, provides 2D images of 3D anatomical structures. Currently, 3D information can be derived using advanced imaging modalities including computed tomography, magnetic resonance imaging, and 3D ultrasound.

### SUMMARY

This document provides materials and methods for determining three-dimensional spatial orientations of blood vessels, cardiac valves, and other anatomical structures within a mammal during a clinical procedure (e.g., an invasive clinical procedure). For example, this document provides balloon catheters with radio-opaque markers and devices that may be either fully or partially radio-lucent (e.g., preferentially transmits x-rays compared to soft tissue) or radio-opaque (e.g., preferentially absorbs x-rays compared to soft tissue) for determining three-dimensional spatial orientations of blood vessels, cardiac valves, and other anatomical structures within a mammal during a clinical procedure. This document also provides methods for using balloon catheters with radio-opaque markers, fully radio-lucent devices, partially radio-lucent devices, and/or radio-opaque devices to determine three-dimensional spatial orientations of blood vessels, cardiac valves, and other anatomical structures within a mammal during a clinical procedure. Examples of balloon catheters that may be used with such methods can include balloon catheters that are custom configured with radio-opaque markers and balloon catheters with radio-opaque markers that are commercially available.

As described herein, a balloon catheter with radio-opaque markers can be inflated within a heart valve, a vessel, or an anatomical structure of interest. While the balloon catheter is inflated, one or more X-ray images of the inflated balloon can be acquired. The acquired X-ray images can be used to calculate the three-dimensional location and orientation of the balloon based on the known X-ray system geometry and the location of the radio-opaque markers of the balloon catheter on the X-ray images. In some cases, a balloon catheter provided herein can be used to determine the three-dimensional spatial location, orientation, and size of a cardiac valve or vessel in which the catheter balloon is inflated. In some cases, a balloon catheter provided herein can be used to determine the three-dimensional spatial location, orientation, and size of a patent foramen ovale or other hole defect in a heart. In some cases, a balloon catheter provided herein can be used to determine the optimum angular orientation of an interventional X-ray system being used to provide guidance during a cardiovascular procedure (e.g., a cardiovascular interventional procedure). In some cases, a balloon catheter provided herein can be used to provide three-dimensional spatial information to a system such as an x-ray system, treatment planning system or a robotic surgical system.

Having the ability to determine three-dimensional spatial orientations of cardiac valves, blood vessels, and other anatomical structures within a mammal during a clinical procedure can provide surgeons with improved precision for localization of trans-catheter valves and stents, can reduce the use of iodine contrast and reduce radiation doses during trans-catheter cardiac valve therapy, and/or can reduce the overall time needed to perform a clinical procedure.

In general, one aspect of this document features a method for determining the three-dimensional angular orientation of a balloon catheter within a target anatomy of a mammal, wherein the balloon catheter comprises one or more rings of radio-opaque markers located around the circumference of the balloon catheter. The method comprises, or consists essentially of, (a) inflating the balloon catheter within the target anatomy, (b) obtaining an X-ray image of the balloon catheter in an inflated configuration under conditions wherein the location of an X-ray source used to obtain the first X-ray image and the image receptor plane of the first X-ray image are known, and (c) calculating the three-dimensional angular orientation of the balloon catheter at the time of the first X-ray image using the X-ray image, the known location of the X-ray source, and the known image receptor plane. The mammal can be a human. The balloon catheter can be a balloon catheter with two to five of the rings. The balloon catheter can be a balloon catheter with two of the rings. The target anatomy can be a blood vessel or cardiac valve. The method can comprise determining an angular projection for the X-ray source that results in an X-ray image that is perpendicular or substantially perpendicular to the target anatomy. The method can comprise determining two or more angular projections for the X-ray source that result in X-ray images that are perpendicular or substantially perpendicular to the target anatomy. The method can comprise determining angular projections, at least 180 degrees around the mammal, for the X-ray source that result in X-ray images that are perpendicular to, parallel to, or at some specified oblique angle with respect to the target anatomy. The method can comprise determining angular projections, 360 degrees around the mammal, for the X-ray source that result in X-ray images that are perpendicular or substantially perpendicular to the target anatomy. The method can comprise obtaining more than one X-ray image of the balloon catheter in an inflated configuration under conditions wherein the location of an X-ray source used to obtain each of the more than one X-ray image and the image receptor plane of each of the more than one X-ray image are known.

In another aspect, this document features a method for determining one or more locations for positioning an X-ray source to obtain an X-ray image that is perpendicular or substantially perpendicular to a target anatomy within a mammal. The method comprises, or consists essentially of, (a) inflating a balloon catheter comprising two or more rings of radio-opaque markers located around the circumference of the balloon catheter within the target anatomy, (b) obtaining a first X-ray image of the balloon catheter in an inflated configuration under conditions wherein the location of an X-ray source used to obtain the first X-ray image and the image receptor plane of the first X-ray image are known, (c) calculating the position of the balloon catheter at the time of the first X-ray image using the first X-ray image, the known location of the X-ray source, and the known image receptor plane, and (d) determining one or more angular projections for the X-ray source around or at least partially around the mammal that result in an X-ray image that is perpendicular or substantially perpendicular to the target anatomy. The mammal can be a human. The balloon catheter can be a balloon catheter with two to five of the rings. The balloon catheter can be a balloon catheter with two of the rings. The target anatomy can be a blood vessel or cardiac valve. The method can comprise determining one angular projection for the X-ray source that results in an X-ray image that is perpendicular or substantially perpendicular to the target anatomy. The method can comprise determining two or more angular projections for the X-ray source that result in an X-ray image that is perpendicular or substantially perpendicular to the target anatomy. The method can comprise determining each angular projection, at least 180 degrees around the mammal, for the X-ray source that results in an X-ray image that is perpendicular or substantially perpendicular to the target anatomy. The method can comprise determining each angular projection, 360 degrees around the mammal, for the X-ray source that results in an X-ray image that is perpendicular or substantially perpendicular to the target anatomy. The method can comprise obtaining more than one X-ray image of the balloon catheter in an inflated configuration under conditions wherein the location of an X-ray source used to obtain each of the more than one X-ray image and the image receptor plane of each of the more than one X-ray image are known. The method can comprise positioning the X-ray source at the one or more angular projections for the X-ray source around or at least partially around the mammal that result in an X-ray image that is perpendicular or substantially perpendicular to the target anatomy. The method can comprise obtaining an X-ray image after the positioning.

In one general aspect, a method is provided for determining the three-dimensional angular orientation of a balloon catheter within a target anatomy of a mammal, wherein said balloon catheter comprises one or more rings of radio-opaque markers located around the circumference of said balloon catheter. The method comprises inflating said balloon catheter within said target anatomy, obtaining an X-ray image of said balloon catheter in an inflated configuration under conditions wherein the location of an X-ray source used to obtain said first X-ray image and the image receptor plane of said first X-ray image are known, and calculating said three-dimensional angular orientation of said balloon catheter at the time of said first X-ray image using said X-ray image, the known location of said X-ray source, and the known image receptor plane.

In various implementations, said mammal may be a human. Said balloon catheter may be a balloon catheter with two to five of said rings. Said balloon catheter may be a balloon catheter with two of said rings. Said target anatomy may be a blood vessel or cardiac valve. Said method may comprise determining an angular projection for said X-ray source that results in an X-ray image that is perpendicular or substantially perpendicular to said target anatomy. Said method may comprise determining two or more angular projections for said X-ray source that result in X-ray images that are perpendicular or substantially perpendicular to said target anatomy. Said method may comprise determining angular projections, at least 180 degrees around said mammal, for said X-ray source that result in X-ray images that are perpendicular or substantially perpendicular to said target anatomy. Said method may comprise determining angular projections, 360 degrees around said mammal, for said X-ray source that result in X-ray images that are perpendicular or substantially perpendicular to said target anatomy. Said method may comprise obtaining more than one X-ray image of said balloon catheter in an inflated configuration under conditions wherein the location of an X-ray source used to obtain each of said more than one X-ray image and the image receptor plane of each of said more than one X-ray image are known.

In another general aspect, a method is provided for determining one or more locations for positioning an X-ray source to obtain an X-ray image that is perpendicular or substantially perpendicular to a target anatomy within a mammal. Said method comprises inflating a balloon catheter comprising two or more rings of radio-opaque markers located around the circumference of said balloon catheter within said target anatomy, obtaining a first X-ray image of said balloon catheter in an inflated configuration under conditions wherein the location of an X-ray source used to obtain said first X-ray image and the image receptor plane of said first X-ray image are known, calculating the position of said balloon catheter at the time of said first X-ray image using said first X-ray image, the known location of said X-ray source, and the known image receptor plane, and determining one or more angular projections for said X-ray source around or at least partially around said mammal that result in an X-ray image that is perpendicular or substantially perpendicular to said target anatomy.

In various implementations, said mammal may be a human. Said balloon catheter may be a balloon catheter with two to five of said rings. Said balloon catheter may be a balloon catheter with two of said rings. Said target anatomy may be a blood vessel or cardiac valve. Said method may comprise determining one angular projection for said X-ray source that results in an X-ray image that is perpendicular or substantially perpendicular to said target anatomy. Said method may comprise determining two or more angular projections for said X-ray source that result in an X-ray image that is perpendicular or substantially perpendicular to said target anatomy. Said method may comprise determining each angular projection, at least 180 degrees around said mammal, for said X-ray source that results in an X-ray image that is perpendicular or substantially perpendicular to said target anatomy. Said method may comprise determining each angular projection, 360 degrees around said mammal, for said X-ray source that results in an X-ray image that is perpendicular or substantially perpendicular to said target anatomy. Said method may comprise obtaining more than one X-ray image of said balloon catheter in an inflated configuration under conditions wherein the location of an X-ray source used to obtain each of said more than one X-ray image and the image receptor plane of each of said more than one X-ray image are known. Said method may comprise positioning said X-ray source at said one or more angular projections for said X-ray source around or at least partially around said mammal that result in an X-ray image that is perpendicular or substantially perpendicular to said target anatomy. Said method may comprise obtaining an X-ray image after said positioning.

In another general aspect, a method is provided for determining the three-dimensional angular orientation of a device within a target anatomy of a mammal, wherein said device can be observed by x-ray imaging and is of known size or shape. Said method comprises delivering said device to said target anatomy, obtaining an X-ray image of said device under conditions wherein the location of an X-ray source used to obtain said first X-ray image and the image receptor plane of said first X-ray image are known, and calculating said three-dimensional angular orientation of said device at the time of said first X-ray image using said X-ray image, the known location of said X-ray source, and the known image receptor plane.

In various implementations, said mammal may be a human. Said device may be a balloon catheter inflated with a contrast medium. Said device may be a structure made of a metal. Said target anatomy may be a blood vessel or cardiac valve. Said method may comprise determining an angular projection for said X-ray source that results in an X-ray image that is perpendicular or substantially perpendicular to said target anatomy. Said method may comprise determining two or more angular projections for said X-ray source that result in X-ray images that are perpendicular or substantially perpendicular to said target anatomy. Said method may comprise determining angular projections, at least 180 degrees around said mammal, for said X-ray source that result in X-ray images that are perpendicular or substantially perpendicular to said target anatomy. Said method may comprise determining angular projections, 360 degrees around said mammal, for said X-ray source that result in X-ray images that are perpendicular or substantially perpendicular to said target anatomy. Said method may comprise obtaining more than one X-ray image of said device under conditions wherein the location of an X-ray source used to obtain each of said more than one X-ray image and the image receptor plane of each of said more than one X-ray image are known.

In another general aspect, a method is provided for determining one or more locations for positioning an X-ray source to obtain an X-ray image that is perpendicular or substantially perpendicular to a target anatomy within a mammal. Said method comprises delivering a device within said target anatomy, obtaining a first X-ray image of said device under conditions wherein the location of an X-ray source used to obtain said first X-ray image and the image receptor plane of said first X-ray image are known, calculating the position of said device at the time of said first X-ray image using said first X-ray image, the known location of said X-ray source, and the known image receptor plane, and determining one or more angular projections for said X-ray source around or at least partially around said mammal that result in an X-ray image that is perpendicular or substantially perpendicular to said target anatomy.

In various implementations, said mammal may be a human. Said device may be a balloon catheter inflated with a contrast medium. Said device may be a structure made of a metal. Said target anatomy may be a blood vessel or cardiac valve. Said method may comprise determining one angular projection for said X-ray source that results in an X-ray image that is perpendicular or substantially perpendicular to said target anatomy. Said method may comprise determining two or more angular projections for said X-ray source that result in an X-ray image that is perpendicular or substantially perpendicular to said target anatomy. Said method may comprise determining each angular projection, at least 180 degrees around said mammal, for said X-ray source that results in an X-ray image that is perpendicular or substantially perpendicular to said target anatomy. Said method may comprise determining each angular projection, 360 degrees around said mammal, for said X-ray source that results in an X-ray image that is perpendicular or substantially perpendicular to said target anatomy. Said method may comprise obtaining more than one X-ray image of said device under conditions wherein the location of an X-ray source used to obtain each of said more than one X-ray image and the image receptor plane of each of said more than one X-ray image are known.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a side view of a valvuloplasty balloon that includes radio-opaque markers in the form of three rings of markers according to some embodiments provided herein.

FIG. 2 is a front view of the valvuloplasty balloon of FIG. 1.

FIG. 3 is a graph showing the position of a source of an X-ray beam and the position of radio-opaque markers of a valvuloplasty balloon located in three-dimensional space. The balloon is tipped 45 degrees and rotated 45 degrees.

FIG. 4 is a graph showing the projection of the radio-opaque makers onto an imaging plane for the radio-opaque markers of the valvuloplasty balloon shown in FIG. 3.

FIG. 5 is a graph showing a projection image of two rings of radio-opaque markers for X-ray system gantry angles that are perpendicular to the length of the balloon.

FIG. 6 is a photograph of a stylized phantom to simulate a valvuloplasty balloon that includes radio-opaque markers forming a ring around the periphery of the phantom.

FIG. 7 is a fluoroscopic store monitor image of the stylized marker phantom of FIG. 6.

FIG. 8 is a graph plotting experimentally (large points) and analytically (small points) determined LR and CC angles that result in x-ray projection that is parallel to the plane of the marker rings and perpendicular to the length of the stylized balloon phantom. The analytical data was calculated from a single store monitor image of the stylized phantom of FIG. 6.

FIG. 9 is a photograph of a balloon device that includes radio-opaque markers in the form of two rings of markers according to some embodiments provided herein.

FIG. 10 is a graph depicting balloon midline orientation angles (θ and φ) and left-right (LR) and cranial-caudal (CC) angles with respect to typical patient orientation for a cardiac catheterization procedure.

FIG. 11 is an X-ray angiographic frame of an inflated balloon acquired using reference projection angles LR_{ref}=CC_{ref}=0°.

FIG. 12 is a graph that illustrates cranial-caudal vs. left-right x-ray system projection angles that are expected to result in x-ray projections which are perpendicular to the long axis of the balloon.

FIG. 13 is a photograph of an X-ray angiographic image of the inflated balloon using projection angles LR=−45° and CC=29° as determined by FIG. 12.

FIG. 14 is a photograph of an X-ray aortagram using projection angles LR=45° and CC=15° as determined by FIG. 12.

FIG. 15 is an X-ray angiographic frame of an inflated commercially available balloon acquired using reference projection angles LR_{ref}=CC_{ref}=0°.

### DETAILED DESCRIPTION

This document provides materials and methods for determining three-dimensional spatial orientations of blood vessels, cardiac valves, and other anatomical structures within a mammal during a clinical procedure (e.g., an invasive clinical procedure). For example, this document provides balloon catheters with radio-opaque markers for determining three-dimensional spatial orientations of blood vessels, cardiac valves, and other anatomical structures within a mammal during a clinical procedure. This document also provides methods for using balloon catheters with radio-opaque markers to determine three-dimensional spatial orientations of blood vessels, cardiac valves, and other anatomical structures within a mammal during a clinical procedure. Examples of balloon catheters that may be used with such methods can include balloon catheters that are custom configured with radio-opaque markers, and balloon catheters with radio-opaque markers that are commercially available. One example of a commercially available balloon catheter with radio-opaque markers that can be used with the methods provided herein is a Nucleus-X valvuloplasty balloon catheter by B. Braun Interventional Systems Inc. (Bethlehem, Pa.) which contains three radio-opaque markers along the length of the catheter of the balloon.

This document also provides devices that may be either fully or partially radio-lucent or radio-opaque for determining three-dimensional spatial orientations of blood vessels, cardiac valves, and other anatomical structures within a mammal during a clinical procedure. In addition, this document provides methods for using such devices to determine three-dimensional spatial orientations of blood vessels, cardiac valves, and other anatomical structures within a mammal during a clinical procedure. For example, the device can be a balloon that is inflated with a contrast medium which is radio-opaque or radio-lucent. In some cases, the device can be a device that includes or is made of a metal or similar material. Any appropriate balloon catheter (e.g., angioplasty or valvuloplasty balloons) can be configured to include one or more rings of radio-opaque markers, or other specified configurations of radio-opaque markers. For example, transluminal balloon catheter such as those provided commercially by B. Braun, Boston Scientific (Natick, Mass.), Cordis (Miami, Fla.), Medtronic Inc. (Minneapolis, Minn.), or Abbott Vascular (Santa Clara, Calif.) can be configured to include one or more rings of radio-opaque markers. While rings of radio-opaque markers are used as an example herein, other known configurations of radio-opaque markers also can be used as an alternative to, or in addition to, ring configurations.

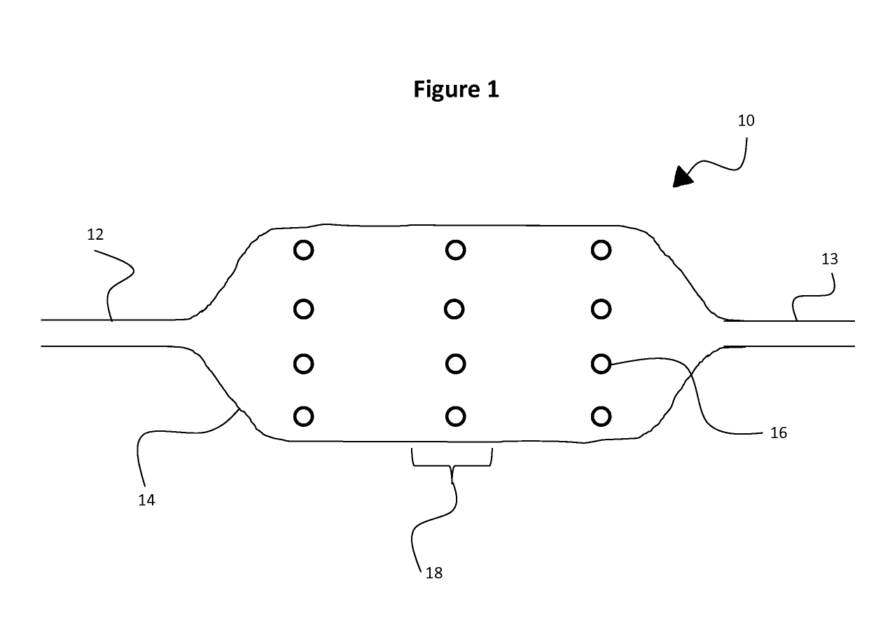

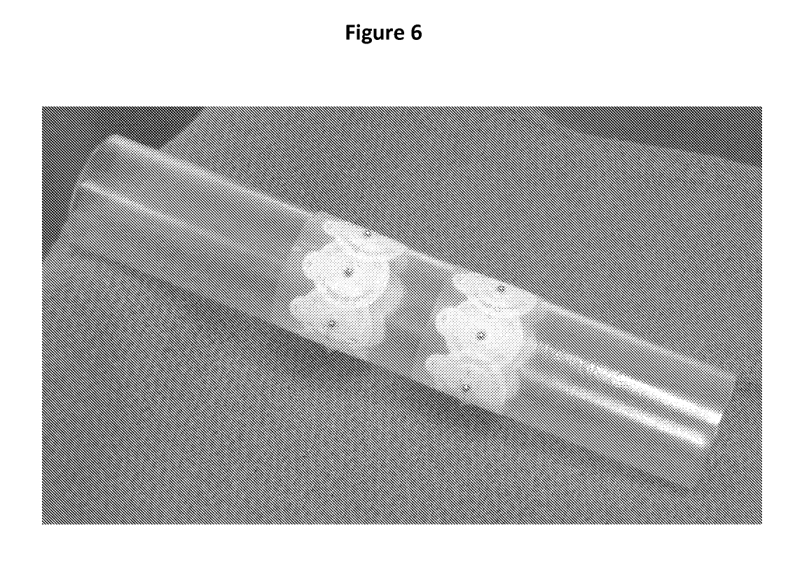

A balloon catheter provided herein can include one or multiple (e.g., two, three, four, five, six, seven, eight, or more) rings of radio-opaque markers. For example, a balloon catheter provided herein can include three rings of radio-opaque markers as shown in FIG. 1 or can contain two rings of radio-opaque markers as shown in the stylized phantom of FIG. 6, which was designed to simulate a valvuloplasty balloon. In some cases, a ring of radio-opaque markers of a balloon catheter provided herein can be a solid continuous ring of radio-opaque material positioned around the circumference of the balloon. In some cases, a ring of radio-opaque markers of a balloon catheter provided herein can be a discontinuous ring of radio-opaque material that is positioned as a series of points around the circumference of the balloon (see, e.g., FIGS. 1, 2, and 6). Any appropriate number of spots of radio-opaque material can be used to form a particular discontinuous ring of radio-opaque material. For example, between about three and 20 spots (e.g., from 3 to 15, from 3 to 10, from 5 to 20, from 5 to 10, or from 10 to 15 spots) can be positioned around the circumference a balloon catheter to form a discontinuous ring of radio-opaque material. In some cases, a balloon catheter provided herein can include two or more rings of radio-opaque markers that are configured such that each ring is parallel or substantially parallel with the other rings. In some cases, the distance between each of the two or more rings of radio-opaque markers that are configured such that each ring is parallel or substantially parallel with the other rings can be between about 2 mm and 30 mm (e.g., from 2 mm to 25 mm, from 2 mm to 20 mm, from 2 mm to 15 mm, from 2 mm to 10 mm, from 5 mm to 30 mm, from 10 mm to 30 mm, or from 5 mm to 15 mm) when the balloon catheter is not in an inflated configuration. For example, the distance between parallel or substantially parallel rings of a balloon catheter provided herein can be about 5 mm when the balloon catheter is not in an inflated configuration.

Any appropriate radio-opaque material can be used to form the rings of radio-opaque markers described herein. For example, stainless steel, cobalt-chromium alloys, tantalum, titanium, titanium alloys, or combinations thereof can be used to form the radio-opaque markers described herein.

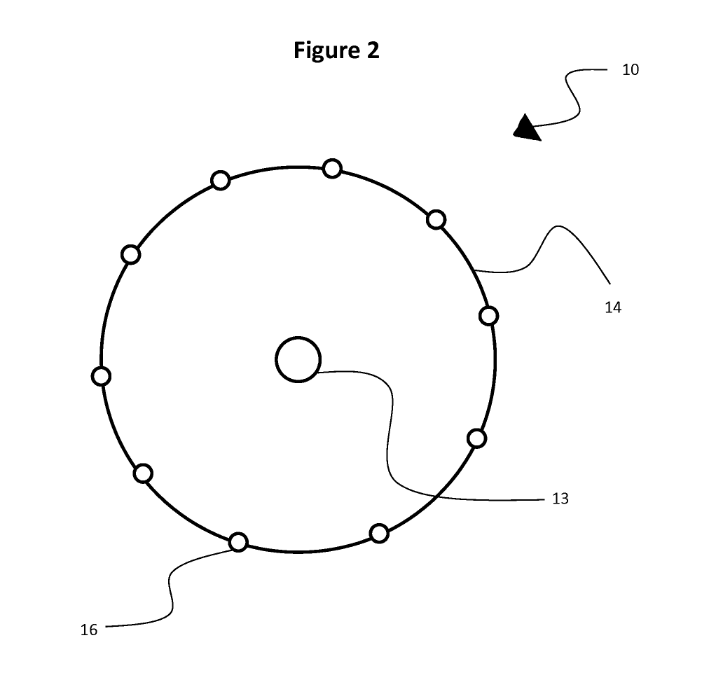

With reference to FIGS. 1 and 2, a balloon catheter 10 can include a proximal end region 12, a distal end region 13, and an inflatable balloon region 14 located between proximal end region 12 and distal end region 13. Inflatable balloon region 14 can include radio-opaque markers 16. Radio-opaque markers 16 can be arranged into one or more rings 18 around the circumference of inflatable balloon region 14. For example, a balloon catheter can have three rings of radio-opaque markers as shown in FIG. 1.

As described herein, analytical techniques can be used in combination with the one or more rings of radio-opaque markers of a balloon catheter provided herein to determine the three-dimensional angular orientations of the inflated balloon catheter itself and thereby the spatial locations of blood vessels, cardiac valves, and other anatomical structures in which the balloon is inflated. The angular orientation of a device in vivo may be determined by image-based analytical methods which use the known size and/or shape of the device, an x-ray image(s) of that device in vivo, and the geometrical properties of the x-ray imaging system as input variables. The angular orientation thus calculated can be used to determine the x-ray system projection angles which result in the x-ray beam travelling parallel to, perpendicular to, or at a specified oblique angle with respect to the orientation of the device and the anatomy that the device is deployed within. For example, a balloon catheter with radio-opaque markers can be inflated within a heart valve, a vessel, or an anatomical structure of interest. While the balloon catheter is inflated, one or more X-ray images of the inflated balloon can be acquired. The acquired X-ray images can be used to calculate the three-dimensional location and orientation of the balloon based on the known X-ray system geometry and the location of the radio-opaque markers of the balloon catheter on the X-ray images.

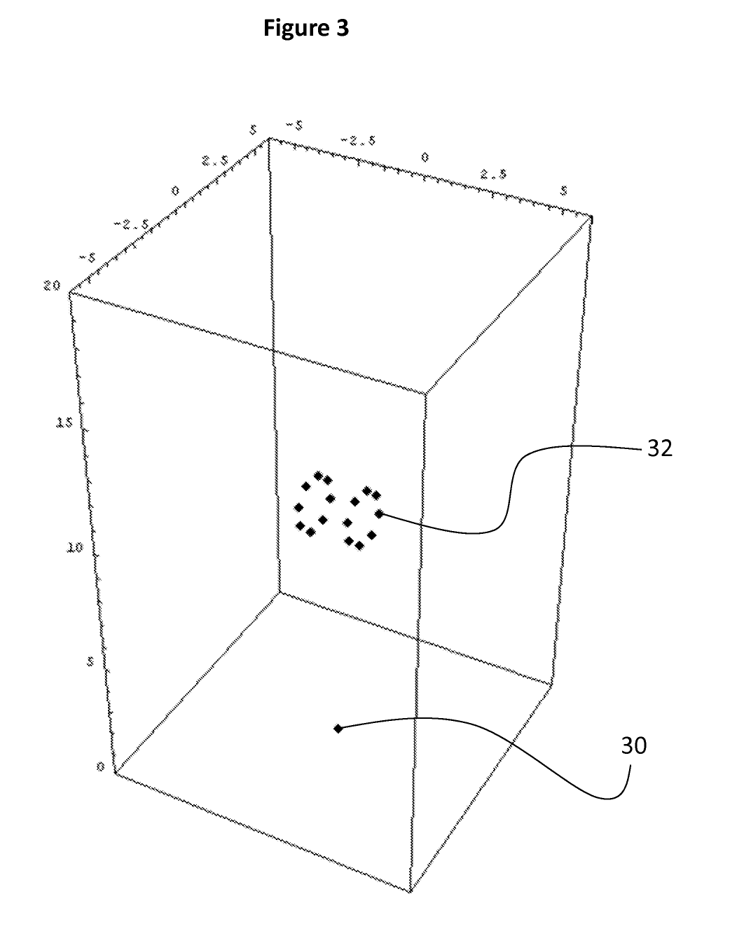

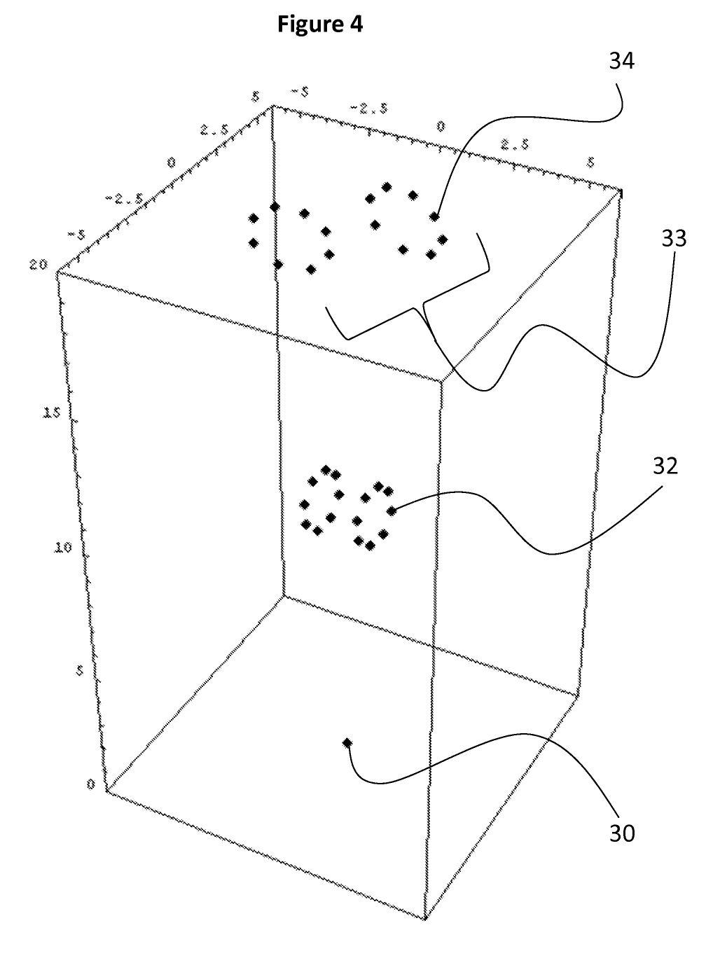

With reference to FIG. 3, a three-dimensional graph can include the known location of an X-ray source 30 (position 0, 0, 0). Prior to obtaining an X-ray image, the three-dimensional angular orientation and spatial location of a balloon catheter containing radio-opaque markers 32 would be unknown. As shown in FIG. 4, an X-ray image 33 can be obtained having spots 34 corresponding to particular radio-opaque markers 32. Using the location of radio-opaque markers 34 of X-ray image 33 for each ring and the known location of X-ray source 30 with respect to the plane containing the X-ray image, the unique three-dimensional angular orientation and spatial location of the balloon catheter containing radio-opaque markers 32 can be determined.

In some cases, three-dimensional angular orientation and spatial location of a balloon catheter containing radio-opaque markers can be determined using various techniques. An example of one such technique is as follows:

1. At first, consider each of N rings of markers independently. Identify the image location of each marker of a ring. The markers from a single ring projected onto the image plane can form a nearly elliptical shape. Analytically determine the location of the center of the ellipse within the image.

2. Computationally construct a virtual plane that is intersected by a ray drawn from the X-ray source to the center of the ellipse. The distance of the virtual plane from the X-ray source can be considered variable, however, description of the analytical process assumes that this distance is equal to the distance between the X-ray source and image plane when the virtual plane is parallel to the image plane.

3. Allow the plane to tip around the center of the ellipse of the image in both the x,z and y,z directions (a_{xz}, a_{yz}) over a range −90 degrees to 90 degrees with respect to the image plane in small increments.

4. Consider a vector extending from the X-ray source through the image plane location of each radio-opaque marker. Identify the (x, y, z) coordinates of the intersection of each vector with each tipped virtual plane.

5. For each tipped virtual plane, calculate the radial distance from the (x, y, z) center of the set of intersection points (same point as the center of the projected ring image in step 1).

6. Determine whether the 3D location of the points of intersection of the vectors and the tipped plane could define a circular ring of markers in (x, y, z) space. For each tipped plane, calculate the standard deviation of the radial distances in step 4. The tip angles (a_{xz}, a_{yz}) that correspond to minimum standard deviations of the radial distances may define the correct plane containing the ring. For a given ring of markers, there are generally two planes in the x,z and y,z space that demonstrate local minima.

7. Repeat above steps for the projected image of the Nth ring(s).

8. For each of N rings, combine the standard deviation values for each tip angle (a_{xz}, a_{yz}) by quadrature sum (square root of the sum of the squared values). The x,z and y,z plane for which this product is minimum defines the angle (a_{xz}, a_{yz}) of the planes containing the physical rings of markers. This angular plane is perpendicular to the length of the balloon.

Another example of a technique for determining the three-dimensional angular orientation and spatial location of a device is as follows:

1. A device or portions thereof may be either radio-lucent (e.g., preferentially transmits x-rays compared to soft tissue) or radio-opaque (e.g., preferentially absorbs x-rays compared to soft tissue), or the device may include radio-opaque markers in a known configuration. For example, the device can be a balloon that is inflated with a contrast medium which is radio-opaque or radio-lucent. In some cases, the device can include a structure made of a metal or similar material. In some cases, the device can be a commercially available valvuloplasty balloon with a known configuration of radio-opaque markers (e.g., B-Braun Nucleus-X balloon).

2. The device or portions of the device can be observed by x-ray imaging.

3. The size and/or shape of the device is known.

4. The device can be positioned or deployed within a cardiovascular structure or other anatomical structure. A balloon device can be inflated therein.

5. The angular orientation of the device in vivo may be specified by the standard spherical coordinate angles θ and φ.

6. The x-y image coordinates of the device or portions of the device can be determined by automated computational methods and/or manually by a human.

7. Based on the x-y image coordinates of the device and a prior knowledge of the size and/or shape of the device, the angles ρ and φ may be determined using analytical methods.

8. Based on θ and φ, x-ray imaging system projection angles (typically cranial-caudal and left-right with respect to the patient) which result in the x-ray beam travelling parallel to, perpendicular to, or at some oblique angle with respect to the orientation of the device and the anatomy that the device is deployed within can be calculated. For example, in the case of the B-Braun Nucleus-X balloon which contains three radio-opaque markers along the length of the catheter of the balloon, θ and φ can be calculated directly from an image of this un-modified COTS catheter.

Other analytical techniques can be performed using the known location of the X-ray source and one or more acquired X-ray images to determine the three-dimensional angular orientation and spatial location of a balloon catheter containing radio-opaque markers.

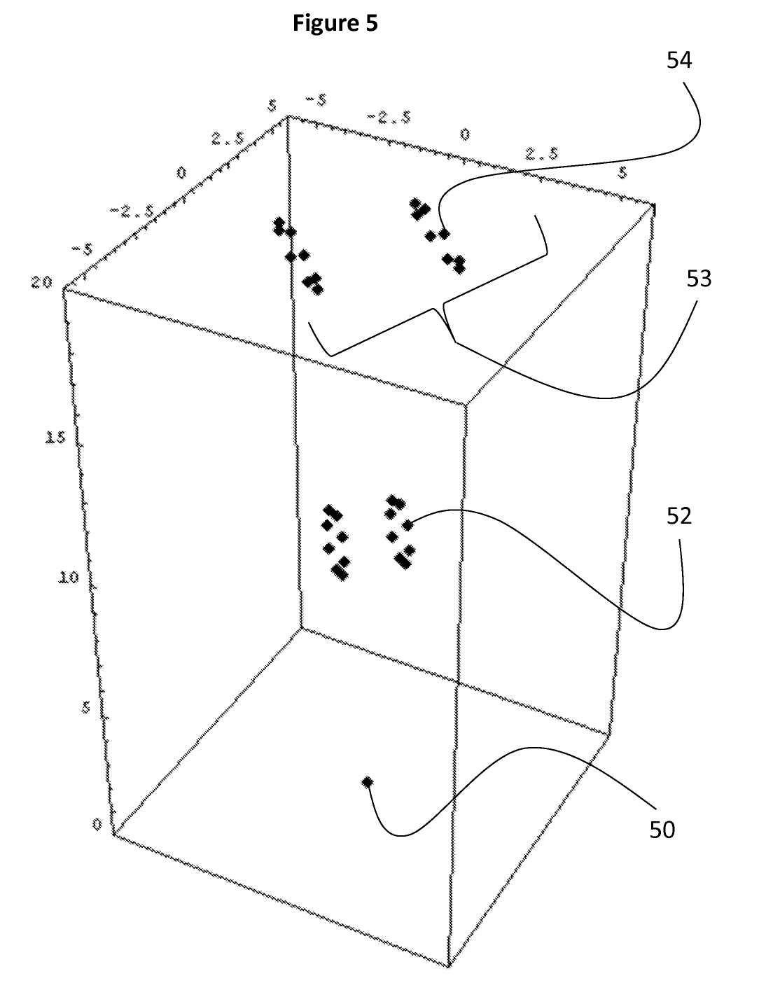

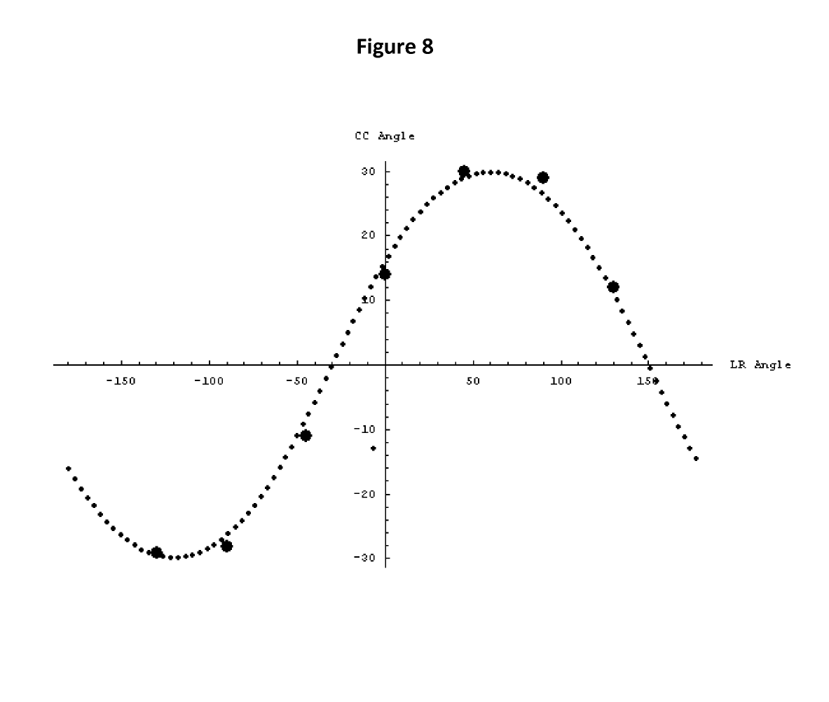

In some cases, the methods and materials provided herein can be used to determine the three-dimensional angular orientation and spatial location of an inflated balloon catheter containing radio-opaque markers. Based on that information, the positioning of an X-ray source can be determined such that a series of additional X-ray images can be obtained in a manner that is parallel to, perpendicular to, or at a specified oblique angle with respect to or substantially perpendicular to the cavity (e.g., blood vessel) that contained the inflated balloon catheter. For example, the angles (e.g., the left/right and cranial/caudal angels) needed to rotate an X-ray source around or partially around a patient in a manner that results in each X-ray image being perpendicular or substantially perpendicular to the cavity (e.g., blood vessel) that contained the inflated balloon catheter can be determined using the initial information about the three-dimensional angular orientation and spatial location of the inflated balloon catheter containing radio-opaque markers (FIG. 8). When the X-ray system is set to angles that result in a projection angle that is perpendicular to the length of the balloon, then the ratio of the major to minor axis of the ellipse is maximized (FIG. 5).

In some cases, the three-dimensional characterization of an anatomy of interest can be determined using one or more X-ray images of a balloon catheter containing one or more rings of radio-opaque markers. In some cases, three-dimensional precision can be improved using more than one X-ray image and/or more than one rings of radio-opaque markers. When using a balloon catheter containing one ring of radio-opaque markers, the three-dimensional characterization of an anatomy of interest can be determined using at least two X-ray images that are acquired using different X-ray beam projection angles.

In some cases, since it is possible that the three-dimensional orientation of an anatomy of interest can be altered temporally with patient respiratory and/or cardiac motion, the methods and materials provided herein can be used to assess the anatomy in both three dimensions and time. For example, a three-dimensional plus time characterization can be used to correct for motion caused by respiratory and/or cardiac motion. In some cases, such motion can be correlated with an electrocardiogram in the case of cardiac motion or can be correlated with thoracic motion using respiratory monitoring and gating in the case of motion caused by breathing.

The invention will be further described in the following examples, which do not limit the scope of the invention described in the claims.

### EXAMPLES

### Example 1

### Ex Vivo Measurement of the Angular Orientation of a Stylized Test Phantom

A stylized phantom to simulate a balloon containing two rings of radio-opaque markers was created by attaching 1.5 mm metal spheres (Spee-D-Mark, The St. John Companies, Valencia, Calif.) to the outside of an cylindrical acrylic tube with an outside diameter of 25 mm and a wall thickness of about 1 mm (FIG. 6). Two rings of seven markers were fastened to the outer wall of the tube. Markers were equally spaced around the circumference of the tube. The rings of markers were separated by a distance of 30 mm along the length of the tube.

The marker ring phantom was fixed on the patient table of a clinical x-ray angiography system (Artis Zee, Siemens Medical Systems, Germany) and was tipped by an unspecified angle with respect to both the table left-right (LR) and cranial-caudal (CC) directions. A fluoroscopic image sequence of the phantom was acquired, and a single frame “store monitor” image was captured and sent to a computer for analysis.

The single frame image was analyzed to determine the 3D angular orientation of the ring objects. There were several steps to the analysis including:
(a) identifying the x-ray shadows of the markers (2×7=14 markers) in the obtained image,
(b) assigning the x-ray shadow of each of the 14 markers to one of two marker rings,
(c) using the image location of the radio-opaque markers from each ring and known x-ray system geometrical properties to determine the unique 3D angular orientation (theta, phi) of the phantom with respect to the patient table of the x-ray system, and
(d) given (theta, phi) and for LR angles in the range −180 to 180 degrees, calculating the x-ray system CC angles that would result in an x-ray projection that is parallel to the 3D planes which contain the physical marker rings.

With the marker phantom fixed on the table of the x-ray system, a series of LR and CC angles that correspond to the plane of the marker rings was determined experimentally. In this case, the LR angle varied throughout the range −130 to 130 degrees, and the CC angle adjusted manually such that the ellipses created in the images by the ring of markers had minor axis radius equal to zero (the shadows of the markers formed two straight lines of markers in the image). In this manner, angular orientation data pairs (LR, CC) were created to compare with predictions from the analytical method above.

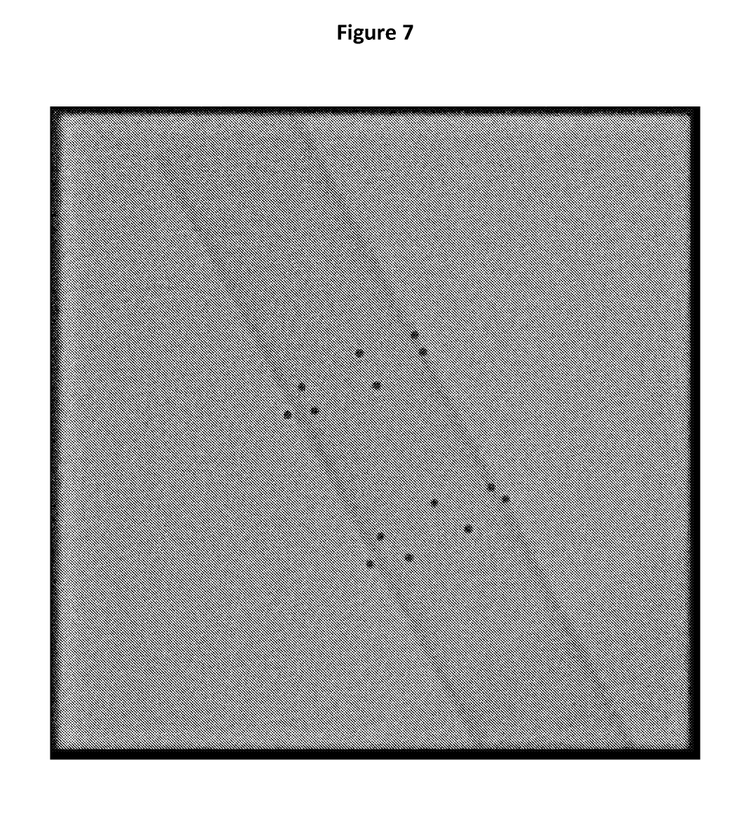

The store monitor X-ray image of the marker phantom is shown in FIG. 7. The angular orientations that would result in a x-ray beam parallel to the 3D planes of the rings of markers are shown in FIG. 8. Both the experimentally determined angles (large points) and analytically determined angles (small points) are shown. In this example, the analytic method indicated that the marker phantom was tipped −76 degrees cranially and 64 degrees with respect to the system left/right orientation. FIG. 8 provides experimental evidence that the analytical methods described herein may be used to determine the 3D angular orientation of a balloon with marker rings and that a continuum of angles (e.g., LR and CC angles) that provide X-ray projection perpendicular to the length of the balloon and parallel to the plane of the rings can be calculated. The ability to calculate the 3D orientation of a tubular object containing two rings of radio-opaque markers was confirmed using a stylized marker phantom. These results demonstrate that angioplasty/valvuloplasty balloons designed to include radio-opaque markers can be used with analytical methods to measure the 3D angular orientation of cardiovascular structures, thereby providing a guide for patient treatment.

### Example 2

### Ex Vivo and In Vivo Validation Using a Custom Balloon with 2 Rings of 6 Radio-Opaque Markers

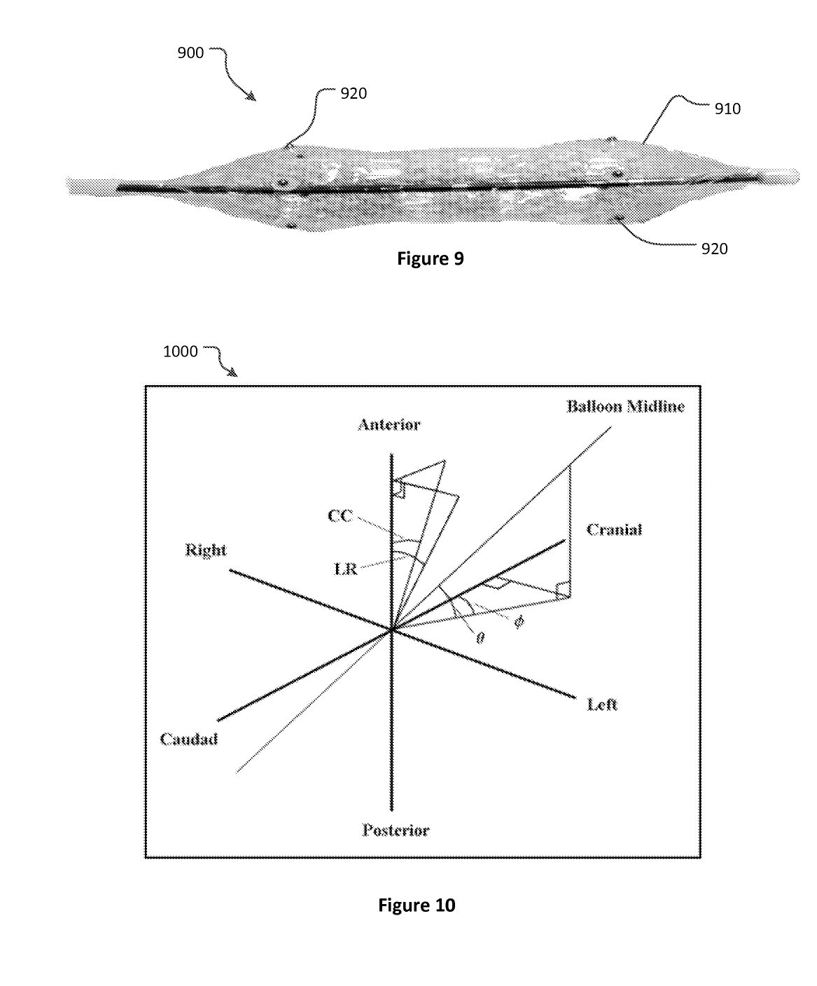

With reference to FIG. 9, the in vivo device 900 used was a valvuloplasty balloon 910 customized to include radio-opaque markers arranged circumferentially around the main body of the balloon 910. The marker balloon device 900 was constructed by securing 1.0 mm diameter tungsten spheres 920 to a 18 mm diameter×40 mm long NuCLEUS-X valvuloplasty balloon 910 (B. Braun Interventional Systems Inc., Bethlehem, Pa.). The spheres 920 were arranged to create 2 rings around the circumference of the balloon 910, with each ring containing 6 spheres. The rings of markers were positioned 17.5 mm from center along the length of the balloon 910.

The angular orientation (θ, φ) of a balloon containing M=2 circular rings each containing N=6 radio-opaque markers was measured from a reference image of the inflated balloon using custom analytical software. To assign 3D angular orientation, consider a default condition in which the midline of the balloon is oriented parallel to the patient head-foot direction of the x-ray system. The angle φ specifies the rotation of the midline of the balloon within a plane parallel to surface of the patient table and the angle θ specifies the angle of rotation the balloon within a plane perpendicular to the plane of the patient table.

An x-ray reference image of the balloon was acquired using x-ray system left-right and cranial-caudal angles LR_{ref} and CC_{ref}. The reference image was analyzed using automated methods to determine the (x_{m,n}, y_{m,n}) image pixel location of each M×N radio-opaque markers. The marker locations were grouped into M=2 groups, each representing one ring of markers. The (xₘ, yₘ) center of mass within the image was calculated for each ring. The angular orientation of the balloon with respect to the patient head-foot direction within the reference image was calculated as:

φ_{ref}=Tan⁻¹[(x₁−x₂)/(y₁−y₂)]. Eq. 1.

Using the center of mass as the virtual origin for each ring of markers, the polar coordinates (r_{m,n}, t_{m,n}) for each of N markers were calculated. To determine the major and minor axes (aₘ and bₘ) of the elliptical shadow of the rings, (r_{m,n}, t_{m,n}) marker locations of each ring were fit to the equation of an ellipse (Mathematica 8.0.4.0, Wolfram Research, Champaign, Ill.),

r_{m,n}=(aₘbₘ)/Sqrt[(aₘ Cos [t_{m,n}−φ_{ref}])²+(bₘ Sin [t_{m,n}−φ_{ref}])²]. Eq. 2.

The angle of rotation of the balloon within the plane which is perpendicular to the x-ray beam was calculated as:

θ_{ref}=Sin⁻¹[a/b], Eq. 3.

where a and b are the average of the minor and major axes of the 2 elliptical shadows of the rings of markers.

Given LR_{ref} and CC_{ref} and with θ_{ref} and φ_{ref} known, x-ray system angles that result in x-ray projections which are perpendicular to the midline of the balloon and (nearly) parallel to the planes of the rings can be calculated. For all θ≠90°, the CC angle which results in x-ray projection perpendicular to the midline of the balloon can be specified for the continuous range of LR angles (−180° to 180°) as:

CC(LR)=Sin⁻¹[Sin [θ] Cos [φ] Cos [ρ]−Sin [φ] Sin [ρ]] for −90°<LR<90°,

CC(LR)=−Sin⁻¹[Sin [θ] Cos [φ] Cos [ρ]−Sin [φ] Sin [ρ]] for all other LR angles, Eq. 4

where

θ=−ArcSin [Cos [θ_{ref}] Sin [LR_{ref}] Sin [φ_{ref}]−Cos [LR_{ref}] (−Cos [φ_{ref}] Cos [θ_{ref}] Sin [CC_{ref}]+Cos [CC_{ref}] Sin [θ_{ref}])], Eq. 5.

φ=ArcTan [(Cos [LR_{ref}] Cos [θ_{ref}] Sin [φ_{ref}]+Sin [LR_{ref}](−Cos [φ_{ref}] Cos [θ_{ref}] Sin [CC_{ref}]+Cos [CC_{ref}] Sin [v]))/(Cos [CC_{ref}] Cos [φ_{ref}] Cos [θ_{ref}]+Sin [CC_{ref}] Sin [θ_{ref}])], Eq. 6.

and

ρ=Tan⁻¹[(−Sin [φ] Sin [θ]−Tan [LR] Cos [θ])/Cos [φ]]. Eq. 7.

For a posterior-anterior x-ray projection where LR_{ref}=CC_{ref}=0°, θ=θ_{ref} and φ=φ_{ref}.

With reference to FIG. 10, graph 1000 depicts balloon midline orientation angles θ and φ that are shown with respect to typical patient orientation for a cardiac catheterization procedure. X-ray system typical left-right (LR) and cranial-caudal (CC) angles are also indicated.

### Ex Vivo Experiments

The balloon was fully inflated with air and secured to the x-ray system table with arbitrary angular orientation (θ, φ). The table of the x-ray system (Artis Zee, Siemens Medical, Erlangen, Germany) was adjusted to ensure that the balloon was at the rotational isocenter. Single frame reference images were acquired for nine LR_{ref} and CC_{ref} projection angles in the range −20° to 20° in 20 degree increments. These frames were used to assess variability of (θ, φ) measurements for a stationary balloon and variable reference projection angles.

### In Vivo Experiments

Experiments were conducted to validate the analytical method and to assess measurement variability in vivo. The marker balloon and analytical process was used to measure the (θ, φ) angular orientation of the LVOT of a pig and then determine the (LR, CC) c-arm angle values that provide x-ray projections which are aligned with the aortic valve plane. The acute pig experiment was performed with approval by our Institutional Animal Care and Use Committee and consistent with Association for Assessment and Accreditation of Laboratory Animal Care guidelines. A 37 kg pig was anesthetized and a 16 fr. introducer sheath was inserted into the femoral artery. The marker balloon was advanced into the left-ventricular outflow tract (LVOT). Ventricular pacing at 280 bpm was used to reduce cardiac function during balloon inflation. The balloon was inflated by manually using a dilution of 1 part 350 mgI/mL Ominipaque™ (iohexol) Injection (GE Heathcare Inc., Princeton, N.J.) and 4 parts saline. All multi-frame acquisition images were acquired using frame rate 15 fps.

During the live experiment, a multi-frame acquisition reference image (I_{ref}) of the inflated balloon was captured using LR_{ref}=CC_{ref}=0°. A single reference frame (F_{ref}) of the multi-frame image was manually selected and used to calculate θ_{ref} and φ_{ref}. These θ_{ref} and φ_{ref} angles were used to calculate subsequent x-ray system projection angles used to validate the methods.

After the live experiment, θ_{ref} and φ_{ref} was measured using several frames from the original multi-frame acquisition reference image (I_{ref}) to assess the variability of θ and φ due to cardiac motion (with rapid pacing of 280 bpm and during balloon inflation). θ_{ref} and φ_{ref} were calculated from 11 consecutive frames (including F_{ref}).

During the live experiment, five (5) x-ray system (LR, CC) angle pairs that are expected provide x-ray projection perpendicular to the long axis of the inflated balloon were calculated using Eq. CC(LR) and LR angles in the range −90° to 90° in 45° increments. For each of these 5 (LR, CC) locations, the balloon was inflated in the LVOT and a multi-frame x-ray image was acquired.

After the live experiment, the 5 images as described in the preceding paragraph were analyzed to determine the relative angular discrepancy between the ideal perpendicular projection angles and the actual projection angles. For these 5 images, it is expected that the x-ray projection was perpendicular to the long axis of the balloon, which would result in an elliptical shadow of the markers with minor axis (a) approaching 0. With a→0, the x-ray shadow of each ring of markers would for a (nearly) straight line in the image. The discrepancy from an ideal perpendicular projection was measured directly as θ_{ref} (Eq. θ ref). Given that the rings of markers are displaced from the center of the balloon by 17.5 mm and that the balloon was 75 cm from the x-ray source, the minimum possible value for θ_{ref} thus measured is 1.3°.

During the live experiment, aortic angiograms were acquired using the same 5 (LR, CC) angles described above. For each angiogram, the heart was allowed to beat normally, 20 ml 350 mgI/mL Omipaque™ was injected at a rate of 10 ml/sec, and images were acquired at a rate of 15 fps.

After the live experiment, the angiograms were evaluated subjectively to assess whether these 5 (LR, CC) angles resulted in x-ray projections which were aligned along the plane of the aortic valve. Specifically, whether the most inferior portions of the aortic valve cusps form a straight line within the image was assessed. Relevant assumptions included that the valve plane is perpendicular to the LVOT and that the angular orientation of the LVOT is not affected by cardiac pacing or balloon inflation (as was used to measure θ and φ).

### Ex Vivo Experiments Results

Considering 9 images acquired over the LR_{ref} and CC_{ref} range −20° to 20°, the average (μ), standard deviation (π), and total angular range (τ) values for θ and φ were μ_{θ}=36.3°, σ_{θ}=0.4°, and τ_{θ}=1.3° and μ_{φ}=35.0°, σ_{φ}=0.7°, and τ_{φ}=1.7°. Overall, variability associated with the balloon and analytical methods is negligible.

### In Vivo Experiments Results

The normal pig heart rate was 90 bpm and the typical average blood pressure was 74 mmHg. During rapid pacing at 280 bpm and balloon inflation, the typical average blood pressure was 35 mmHg. The pig remained clinically stable throughout the experiment. Early in the live experiment, 2 markers disconnected from the balloon. Visual estimation and manual specification of the image locations of the missing markers was performed for subsequent quantitative analysis. Post-experiment evaluation suggested that the error introduced by visual estimation of the location of the missing markers lead to negligible error in θ and φ.

During the live experiment, analysis of a single reference frame (FIG. 3) was used to measure that the balloon was oriented in-vivo at angles θ=29.4° and φ=11.8°. CC angles for the continuous range of LR angles that are expected to result in x-ray projections perpendicular to the long axis of the balloon are indicated by the black line in FIG. 4. The average, standard deviation, and total angular range values for θ and φ, measured throughout the cardiac cycle using 11 consecutive images (nominally 3.4 heart beats), were μ_{θ}=27.9°, σ_{θ}=1.1°, and τ_{θ}=3.5° and μ_{φ}=12.2°, σ_{φ}=0.8°, and τ_{φ}=2.6°. This corresponding range of (LR, CC) values is represented by the gray lines in FIG. 4.

Analysis of image frames containing the inflated balloon and acquired for 5 x-ray system (LR, CC) combinations (circles in FIG. 4) indicated that the specified projection angles deviated from the ideal perpendicular projection angles by μ=2.6 °, σ=1.1°, and τ=3.0°. This average deviation from ideal is inclusive of the 1.3° minimum possible value due to offset of the rings of markers from balloon midline. Overall, the magnitude of error of specified (LR, CC) x-ray angles is consistent with the angular range of the anatomy due to cardiac motion.

Visual inspection of the aortic angiograms acquired for 5 x-ray system (LR, CC) combinations and with the heart beating normally demonstrated that the x-ray beam was well aligned with the plane of the aortic valve (FIG. 6) and that the perception of alignment was variable throughout the normal cardiac cycle. Discrepancy between the x-ray projection angle and valve plane is likely due to multiple factors including: 1) motion associated with normal cardiac function; 2) that the normal pig valve plane was assumed but not known to be perpendicular to the anatomical long axis of the LVOT; 3) that rapid pacing altered the normal angular orientation of the LVOT and valve plane; 4) that the inflated balloon altered the normal orientation of the LVOT and valve plane. In the last case, note that the 40 mm long balloon was positioned within portions in the LV, across the aortic valve, and within the aorta. Use of a shorter balloon may help to reduce possible perturbation of the orientation of the LVOT.

This work demonstrates that the 3D angular orientation of the LVOT of a live pig can be accurately measured using a balloon containing rings of markers and image-based analytical methods. The accuracy of the current method may be improved by considering many reference image frames rather than a single frame to measure θ and φ. While the methods presented here were specific to a balloon with radio-opaque markers, it is anticipated that other balloons or devices could be used in a similar manner provided that the size and/or shape of the balloon or device is known and that the device can be distinguished in a x-ray image and that appropriate analytical methods are developed.

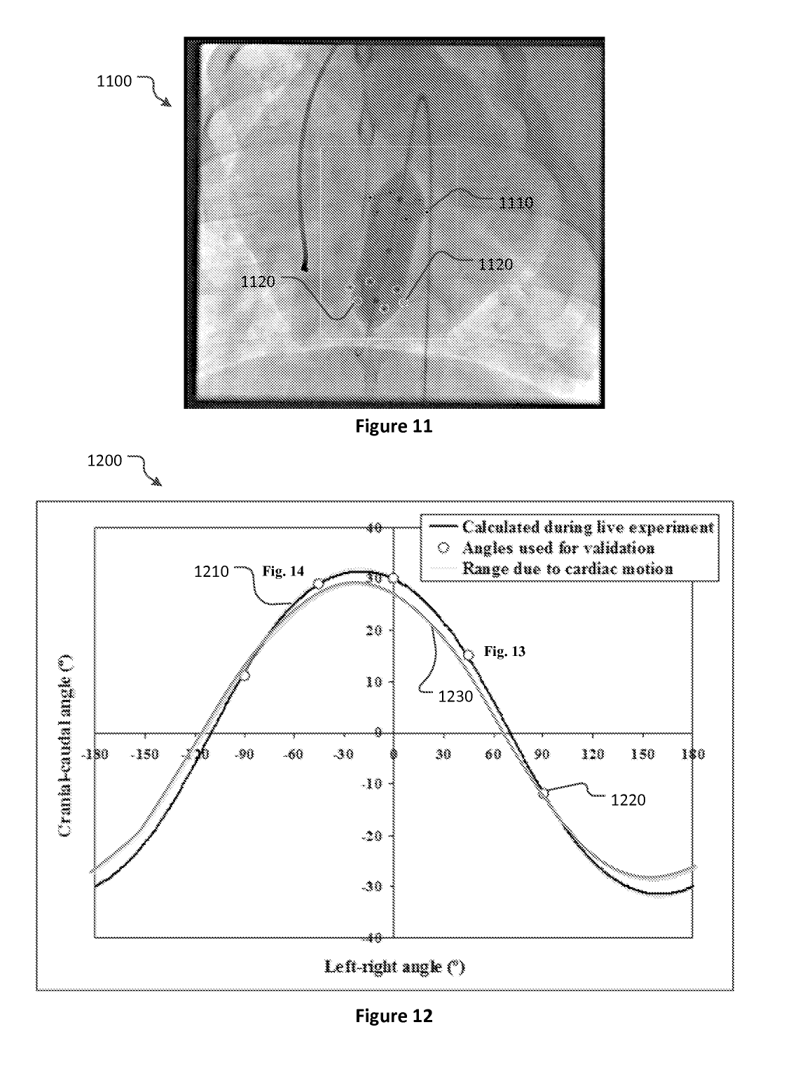

FIG. 11 is an X-ray angiographic frame 1100 of the inflated balloon acquired using reference projection angles LR_{ref}=CC_{ref}=0°. Circles 1110 indicate automatically determined marker locations. The location of missing markers (empty circles 1120) was specified manually by visual estimation.

FIG. 12 is a graph 1200 that illustrates cranial-caudal vs. left-right x-ray system projection angles that are expected to result in x-ray projections which are perpendicular to the long axis of the balloon. The black line 1210 represents the angles calculated during the live experiment; circles 1220 indicate projection angles used to acquire images for validation; and gray lines 1230 represent the range of angles due to cardiac motion during rapid pacing and balloon inflation.

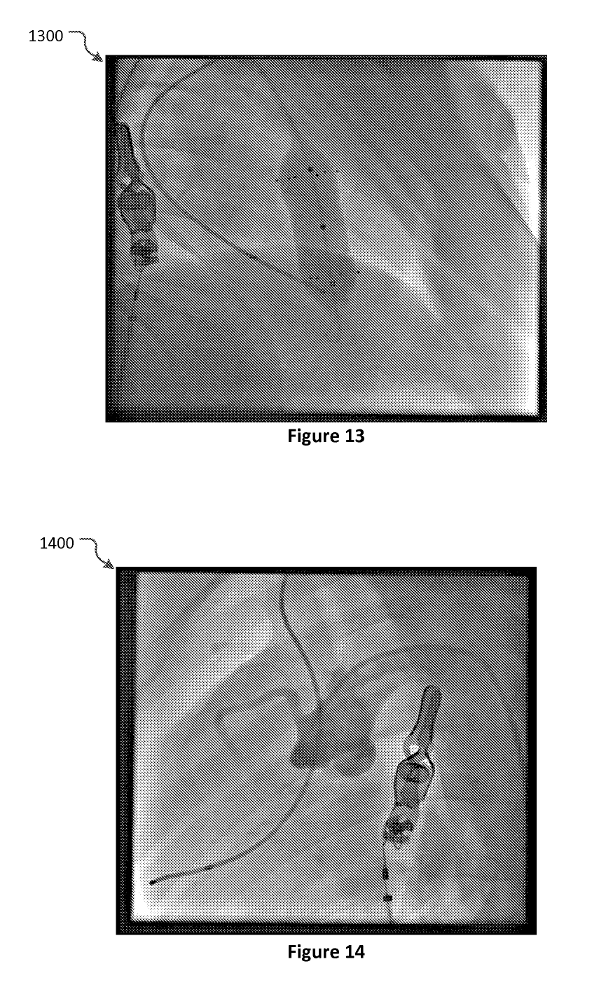

FIG. 13 illustrates X-ray angiographic image 1300 of the inflated balloon using projection angles LR=−45° and CC=29° as determined by FIG. 12. That the x-ray shadows of the markers are arranged in a nearly perfect line indicates that the x-ray projection was aligned with the plane of the ring of markers. Measured discrepancy between the ideal perpendicular projection and actual projection angle was 2.1°.

FIG. 14 is an X-ray aortagram 1400 using projection angles LR=45° and CC=15° as determined by FIG. 12. That the three cusps of the aortic valve are well aligned indicates that the methods accurately predicted that this x-ray projection angle was aligned with the plane of the aortic valve.

### Example 3

### In Vivo Validation of Methods Using a Commercial Valvuloplasty Balloon

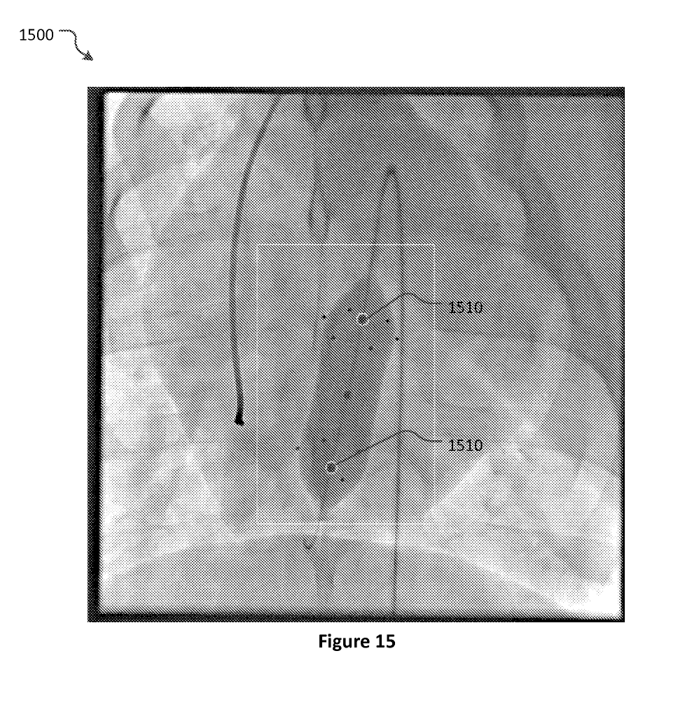

This example used a commercially available valvuloplasty balloon catheter with a known configuration of radio-opaque markers. The following input parameters (or functional equivalents) are required and are readily available:
1) Real-world distance between the ends of the linear device (L, mm)
2) (xₘ, yₘ) image coordinates of the ends of the linear device
3) Pixel pitch of the imaging system (p, mm)
4) X-ray source to device distance (D_{object}, cm) from the DICOM header of the x-ray image when the device is positioned at the rotational isocenter of the imaging system
5) X-ray source to image receptor distance (D_{image}, cm) from the DICOM header of the x-ray image
   Then θ_{ref}=Cos⁻¹(L_{image}/L),
where L_{image}=Sqrt[(x₁²−x₂²)/(y₁²−y₂²)]*p*D_{object}/D_{image},
   and φ_{ref}=Tan⁻¹[(x₁−x₂)/(y₁−y₂)].
      FIG. 15 is an X-ray angiographic frame 1500 of the inflated balloon acquired using reference projection angles LR_{ref}=CC_{ref}=0°. Circles 1510 indicate manually determined locations of the ends of the linear device in-vivo.
      In vivo Experiments Results—Commercial Balloon

The same images were used to measure in vivo orientation of the LVOT using both the custom and commercial balloon methods.

Measurements using the commercial balloon methods differed from the custom balloon measurements by −0.5±0.4°

These results demonstrate that the methods provided herein can be used to measure accurately the 3D angular orientation of the LVOT of a live mammal by analyzing a 2D projection image of a valvuloplasty balloon containing radio-opaque markers. That the angular projection measurements can then be used to select x-ray projection angles that are well aligned with the aortic valve plane was demonstrated. These methods can be used to specify patient-specific x-ray projection angles for valve deployment during TAVI or other invasive cardiovascular procedures for which alignment of the x-ray beam with respect to the 3D angular orientation of the anatomy is desired.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining the three-dimensional angular orientation of a balloon catheter within a target anatomy of a mammal, wherein said balloon catheter comprises one or more rings of radio-opaque markers located around the circumference of said balloon catheter, and wherein said method comprises:
(a) inflating said balloon catheter within said target anatomy,
(b) obtaining an X-ray image of said balloon catheter in an inflated configuration under conditions wherein the location of an X-ray source used to obtain said first X-ray image and the image receptor plane of said first X-ray image are known, and
(c) calculating said three-dimensional angular orientation of said balloon catheter at the time of said first X-ray image using said X-ray image, the known location of said X-ray source, and the known image receptor plane.

2. (canceled)

3. The method of claim 1, wherein said balloon catheter is a balloon catheter with two to five of said rings.

4. (canceled)

5. (canceled)

6. The method of claim 1, wherein said method comprises determining an angular projection for said X-ray source that results in an X-ray image that is perpendicular or substantially perpendicular to said target anatomy.

7. The method of claim 1, wherein said method comprises determining two or more angular projections for said X-ray source that result in X-ray images that are perpendicular or substantially perpendicular to said target anatomy.

8. The method of claim 1, wherein said method comprises determining angular projections, at least 180 degrees around said mammal, for said X-ray source that result in X-ray images that are perpendicular or substantially perpendicular to said target anatomy.

9. The method of claim 1, wherein said method comprises determining angular projections, 360 degrees around said mammal, for said X-ray source that result in X-ray images that are perpendicular or substantially perpendicular to said target anatomy.

10. The method of claim 1, wherein said method comprises obtaining more than one X-ray image of said balloon catheter in an inflated configuration under conditions wherein the location of an X-ray source used to obtain each of said more than one X-ray image and the image receptor plane of each of said more than one X-ray image are known.

11. A method for determining one or more locations for positioning an X-ray source to obtain an X-ray image that is perpendicular or substantially perpendicular to a target anatomy within a mammal, wherein said method comprises:
(a) inflating a balloon catheter comprising two or more rings of radio-opaque markers located around the circumference of said balloon catheter within said target anatomy,
(b) obtaining a first X-ray image of said balloon catheter in an inflated configuration under conditions wherein the location of an X-ray source used to obtain said first X-ray image and the image receptor plane of said first X-ray image are known,
(c) calculating the position of said balloon catheter at the time of said first X-ray image using said first X-ray image, the known location of said X-ray source, and the known image receptor plane, and
(d) determining one or more angular projections for said X-ray source around or at least partially around said mammal that result in an X-ray image that is perpendicular or substantially perpendicular to said target anatomy.

12. (canceled)

13. The method of claim 11, wherein said balloon catheter is a balloon catheter with two to five of said rings.

14. (canceled)

15. (canceled)

16. The method of claim 11, wherein said method comprises determining one angular projection for said X-ray source that results in an X-ray image that is perpendicular or substantially perpendicular to said target anatomy.

17. The method of claim 11, wherein said method comprises determining two or more angular projections for said X-ray source that result in an X-ray image that is perpendicular or substantially perpendicular to said target anatomy.

18. The method of claim 11, wherein said method comprises determining each angular projection, at least 180 degrees around said mammal, for said X-ray source that results in an X-ray image that is perpendicular or substantially perpendicular to said target anatomy.

19. The method of claim 11, wherein said method comprises determining each angular projection, 360 degrees around said mammal, for said X-ray source that results in an X-ray image that is perpendicular or substantially perpendicular to said target anatomy.

20. The method of claim 11, wherein said method comprises obtaining more than one X-ray image of said balloon catheter in an inflated configuration under conditions wherein the location of an X-ray source used to obtain each of said more than one X-ray image and the image receptor plane of each of said more than one X-ray image are known.

21. The method of claim 11, wherein said method comprises positioning said X-ray source at said one or more angular projections for said X-ray source around or at least partially around said mammal that result in an X-ray image that is perpendicular or substantially perpendicular to said target anatomy.

22. (canceled)

23. A method for determining the three-dimensional angular orientation of a device within a target anatomy of a mammal, wherein said device can be observed by x-ray imaging and is of known size or shape, and wherein said method comprises:
(a) delivering said device to said target anatomy,
(b) obtaining an X-ray image of said device under conditions wherein the location of an X-ray source used to obtain said first X-ray image and the image receptor plane of said first X-ray image are known, and
(c) calculating said three-dimensional angular orientation of said device at the time of said first X-ray image using said X-ray image, the known location of said X-ray source, and the known image receptor plane.

24. (canceled)

25. The method of claim 23, wherein said device is a balloon catheter inflated with a contrast medium.

26. The method of claim 23, wherein said device is a structure made of a metal.

27. (canceled)

28. The method of claim 23, wherein said method comprises determining an angular projection for said X-ray source that results in an X-ray image that is perpendicular or substantially perpendicular to said target anatomy.

29. The method of claim 23, wherein said method comprises determining two or more angular projections for said X-ray source that result in X-ray images that are perpendicular or substantially perpendicular to said target anatomy.

30. The method of claim 23, wherein said method comprises determining angular projections, at least 180 degrees around said mammal, for said X-ray source that result in X-ray images that are perpendicular or substantially perpendicular to said target anatomy.

31. The method of claim 23, wherein said method comprises determining angular projections, 360 degrees around said mammal, for said X-ray source that result in X-ray images that are perpendicular or substantially perpendicular to said target anatomy.

32. The method of claim 23, wherein said method comprises obtaining more than one X-ray image of said device under conditions wherein the location of an X-ray source used to obtain each of said more than one X-ray image and the image receptor plane of each of said more than one X-ray image are known.

33-44. (canceled)
